(19) United States
(12) Patent Application Publication
(10) Pub. No.: US 2021/0170409 A1
(43) Pub. Date: Jun. 10, 2021

(54) MICROFLUIDIC CHIP FOR CIRCULATING TUMOR CELL SEPARATION, CIRCULATING TUMOR CELL SEPARATION METHOD AND COUNTING METHOD

(71) Applicant: SOUTH UNIVERSITY OF SCIENCE AND TECHNOLOGY OF CHINA, Guangdong (CN)

(72) Inventors: Xing Cheng, Guangdong (CN); Youwei Jiang, Guangdong (CN); Zhenming Yu, Guangdong (CN); Xinglong Huang, Guangdong (CN); Rifei Chen, Guangdong (CN)

(21) Appl. No.: 16/616,745

(22) PCT Filed: Mar. 16, 2018
(86) PCT No.: PCT/CN2018/079320

§ 371 Date: Nov. 25, 2019

(30) Foreign Application Priority Data
May 25, 2017 (CN) 201710378545.X

(51) Int. Cl. B01L 3/00; C12M 3/06; C12M 1/12; C12N 5/09; G01N 33/50

(52) U.S. Cl. CPC B01L 3/502753; C12M 23/16; C12M 25/02; C12N 5/0694; B01L 2200/0668; B01L 3/502761; B01L 2200/0652; B01L 2300/0681; G01N 33/5005

(57) ABSTRACT

A microfluidic chip for circulating tumor cell separation, comprising a first shell layer, a second shell layer, and a filter membrane between the first shell layer and the second shell layer. A first channel is formed between the filter membrane and the first shell layer; a second channel is formed between the filter membrane and the second shell layer; the first shell layer is provided with m input interfaces and n output interfaces, wherein m is greater than or equal to 1 and n is greater than or equal to 1; the second shell layer is provided with x input interfaces and y output interfaces, wherein x is greater than or equal to 1 and y is greater than or equal to 1. The chip is used for circulating tumor cell separation to achieve high flux, high efficiency, and a simple method, and facilitate promotion.

### TECHNICAL FIELD

The present application belongs to the technical field of cell separation, and relates to a microfluidic chip for separating circulating tumor cells, and to a method for separating circulating tumor cells and a method for counting the same.

### BACKGROUND

Circulating tumor cells, a general term for various kinds of tumor cells that have detached from primary tumor lesions and entered into human peripheral bloodstream, act as a marker for tumor metastasis. Therefore, the capture and detection of circulating tumor cells are of great significance in the diagnosis of early-stage cancer, prognosis analysis, personalized medicine, or single-cell sequencing of tumors. Related studies show that the level of circulating tumor cells in a patient is positively correlated with progression-free survival and overall survival. At present, liquid biopsy of circulating tumor cells has been clinically applied in the diagnosis of colon cancer, breast cancer, and prostate cancer, and shows great application value and promise in the market. The content of circulating tumor cells in the blood is extremely low. The concentration ratio of blood cells to circulating tumor cells in the blood is greater than 10⁶. Therefore, how to effectively separate and enrich circulating tumor cells becomes the focus and challenge of current researches. Methods for separating circulating tumor cells are mainly divided into biochemical methods and physical methods, depending on the separation mechanisms. A representative product which uses a biochemical method is the CellSearch system which is produced by Johnson & Johnson and has been approved by US FDA; and physical methods mainly include filtration, inertial forces, and deterministic lateral displacement.

The mechanism of the CellSearch system to separate circulating tumor cells is that the epithelial cell adhesion molecule specifically presented on the circulating tumor cell wall specifically binds to an antibody; when an antibody modified with magnetic beads is thoroughly mixed with the human peripherally circulating blood, circulating tumor cells will bind to the antibody, and the cell surface will thus be modified by magnetic beads; under the action of magnetic force, the circulating tumor cells modified by magnetic beads will be displaced in the blood sample and thus be separated from blood cells. However, this technology has the following shortcomings: firstly, the expression of epithelial cell adhesion molecules may be low or even none, which leads to a missed detection of circulating tumor cells, thereby fundamentally affecting the accuracy of the biochemical method for detecting circulating tumor cells; secondly, it takes a long time for the epithelial adhesion molecule to specifically bind to the antibody, making the processing of the blood sample slow; thirdly, the blood sample to be processed is in a large amount, and thus a large amount of the antibody is required, resulting in a high cost. The physical methods for separating circulating tumor cells have mainly disadvantages including low purity of separated tumor cells and serious contamination of white blood cells, which eventually leads to false positives in the dyeing process, that is, false detections. The above defects limit the clinical promotion and application of these technologies.

### SUMMARY

In view of the technical problems existing in the prior art, the present application provides a microfluidic chip for separating circulating tumor cells, a method for separating circulating tumor cells by using the microfluidic chip for separating circulating tumor cells, and a method for counting circulating tumor cells by using the microfluidic chip for separating circulating tumor cells. The methods for separating and counting circulating tumor cells have advantages including high throughput, high efficiency, and ease to operate and promote.

To achieve the objects as described above, the present application uses the following technical solutions.

In a first aspect, one of the objects of the present application is to provide a microfluidic chip for separating circulating tumor cells, which comprises a first housing layer, a second housing layer, and a filter membrane disposed between the first housing layer and the second housing layer, wherein a first channel is formed between the filter membrane and the first housing layer, and a second channel is formed between the filter membrane and the second housing layer;
the first housing layer is provided with m inlet(s) and n outlet(s), wherein m≥1 and n≥1;

the second housing layer is provided with x inlet(s) and y outlet(s), wherein x≥1 and y≥1.

Wherein, m may be 1, 2, 3, 4 or 5, etc., n may be 1, 2, 3, 4 or 5, etc., x may be 1, 2, 3, 4 or 5, etc., and y may be 1, 2, 3, 4 or 5, etc. However, they are not limited to those specified above, and other un-specified values within the above ranges are also applicable.

In some specific embodiments, m, n, x, and y are independent of each other. For example, m is 1, n is 1, x is 1, and y is 1; m is 2, n is 1, x is 2, and y is 3; m is 1, n is 2, x is 3, and y is 4; m is 2, n is 2, x is 2, and y is 5; m is 2, n is 2, x is 4, and y is 4; or m is 3, n is 2, x is 5, and y is 5.

In a preferred embodiment of the present application, the material of the first housing layer comprises any one selected from the group consisting of dimethylsiloxane, polymethylmethacrylate, and polycarbonate, or a combination of at least two selected therefrom. Typical but non-limiting examples of such combinations include: a combination of dimethylsiloxane and polymethylmethacrylate, a combination of polymethylmethacrylate and polycarbonate, a combination of polycarbonate and dimethylsiloxane, or a combination of dimethylsiloxane, polymethylmethacrylate, and polycarbonate, etc.

In a preferred embodiment of the present application, the material of the second housing layer comprises any one selected from the group consisting of dimethylsiloxane, polymethylmethacrylate, and polycarbonate, or a combination of at least two selected therefrom. Typical but non-limiting examples of such combinations include: a combination of dimethylsiloxane and polymethylmethacrylate, a combination of polymethylmethacrylate and polycarbonate, a combination of polycarbonate and dimethylsiloxane, or a combination of dimethylsiloxane, polymethylmethacrylate, and polycarbonate, etc.

In a preferred embodiment of the present application, the material of the filter membrane comprises any one selected from the group consisting of dimethylsiloxane, polymethylmethacrylate, and polycarbonate, or a combination of at least two selected therefrom.

Typical but non-limiting examples of such combinations include: a combination of dimethylsiloxane and polymethylmethacrylate, a combination of polymethylmethacrylate and polycarbonate, a combination of polycarbonate and dimethylsiloxane, or a combination of dimethylsiloxane, polymethylmethacrylate, and polycarbonate, etc.

In a preferred embodiment of the present application, the filter membrane has a pore diameter of 7˜15 μm, such as 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, or 14.5 μm, etc. However, the pore diameter is not limited to those specified above, and other un-specified values within the above ranges are also applicable. Preferably, the filter membrane has a pore diameter of 8˜10 μm.

The basic principle of this technology is that circulating tumor cells can be separated by filtering off blood cells from the blood with a lateral-flow microfluidic filtration device based on the fact that circulating tumor cells in the blood are usually larger than blood cells (red blood cells, white blood cells, etc.), and circulating tumor cells can be counted by staining and photographing.

Based on the above principle, the microfluidic chip for separating circulating tumor cells as provided by the present application is designed according to the following principle: allow a blood sample containing circulating tumor cells to flow in the channel on one side and to be filtered through a filter membrane, upon which, blood cells having a smaller volume will pass through the filter membrane and enter into the channel on the other side, along which channel they flow and then be discharged, while circulating tumor cells having a larger volume cannot pass through the filter membrane and will flow with the blood sample in the original channel and then be collected.

In a second aspect, the present application aims to provide a method for separating circulating tumor cells by using the microfluidic chip for separating circulating tumor cells, which comprises the following steps:

(1) opening the inlet(s) of the first housing layer and the outlet(s) of the second housing layer, closing the outlet(s) of the first housing layer and the inlet(s) of the second housing layer, and inputting a blood sample via the inlet(s) of the first housing layer, filtering the same, and discharging the filtrate via the outlet(s) of the second housing layer;

(2) opening the inlet(s) of the second housing layer and the outlet(s) of the second housing layer, closing the outlet(s) of the first housing layer and the inlet(s) of the first housing layer, and inputting a buffer via the inlet(s) of the second housing layer, and discharging outflow via the outlet(s) of the second housing layer;

(3) opening the inlet(s) of the second housing layer and the outlet(s) of the first housing layer, closing the outlet(s) of the second housing layer and the inlet(s) of the first housing layer, and inputting a buffer via the inlet(s) of the second housing layer, and discharging outflow via the outlet(s) of the first housing layer;

(4) opening the inlet(s) of the first housing layer and the outlet(s) of the first housing layer, closing the outlet(s) of the second housing layer and the inlet(s) of the second housing layer, and inputting a buffer via the inlet(s) of the first housing layer, and discharging outflow via the outlet(s) of the first housing layer;

(5) opening the inlet(s) of the first housing layer and the outlet(s) of the second housing layer, closing the outlet(s) of the first housing layer and the inlet(s) of the second housing layer, and inputting a buffer via the inlet(s) of the first housing layer, and discharging outflow via the outlet(s) of the second housing layer.

Wherein, the buffer comprises any one selected from the group consisting of water, phosphate buffer, phosphate buffer added with bovine serum albumin, culture medium, or serum, or a combination of at least two selected therefrom.

In a preferred embodiment of the present application, steps (2) to (5) are successively repeated 1-20 time(s), for example 1 time, 2 times, 3 times, 4 times, 5 times, 6 times, 7 times, 8 times, 9 times, 10 times, 12 times, 15 times, 18 times, or 20 times, etc. However, the repeated times are not limited to those specified above, and other un-specified values within the above ranges are also applicable.

In a third aspect, the present application aims to provide use of the microfluidic chip for separating circulating tumor cells as described in the first aspect or the method as described in the second aspect for separating circulating tumor cells.

In a fourth aspect, the present application aims to provide a method for counting circulating tumor cells by using the microfluidic chip for separating circulating tumor cells, which comprises the following steps:

(1′) opening the inlet(s) of the first housing layer and the outlet(s) of the second housing layer, closing the outlet(s) of the first housing layer and the inlet(s) of the second housing layer, and inputting a blood sample via the inlet(s) of the first housing layer, filtering the same, and discharging the filtrate via the outlet(s) of the second housing layer;

(2′) opening the inlet(s) of the second housing layer and the outlet(s) of the second housing layer, closing the outlet(s) of the first housing layer and the inlet(s) of the first housing layer, and inputting a buffer via the inlet(s) of the second housing layer, and discharging outflow via the outlet(s) of the second housing layer;

(3′) opening the inlet(s) of the first housing layer and the outlet(s) of the second housing layer, closing the outlet(s) of the first housing layer and the inlet(s) of the second housing layer, and inputting a buffer via the inlet(s) of the first housing layer, and discharging outflow via the outlet(s) of the second housing layer;

(4′) opening the inlet(s) of the first housing layer, closing the outlet(s) of the first housing layer, the inlet(s) of the second housing layer, and the outlet(s) of the second housing layer, inputting a dyeing solution via the inlet(s) of the first housing layer until the chip is filled with the dyeing solution, and allowing the chip to stand;

(5′) opening the inlet(s) of the first housing layer and the outlet(s) of the second housing layer, closing the outlet(s) of the first housing layer and the inlet(s) of the second housing layer, and inputting a washing solution via the inlet(s) of the first housing layer, and discharging outflow via the outlet(s) of the second housing layer;

(6′) opening the inlet(s) of the second housing layer and the outlet(s) of the first housing layer, closing the outlet(s) of the second housing layer and the inlet(s) of the first housing layer, and inputting a washing solution via the inlet(s) of the second housing layer, and discharging outflow via the outlet(s) of the first housing layer;

(7′) observing fluorescence signals from the stained cells inside the chip under a fluorescence microscope, and photographing the same for counting circulating tumor cells.

Wherein, the buffer comprises any one selected from the group consisting of water, phosphate buffer, phosphate buffer added with bovine serum albumin, culture medium, or serum, or a combination of at least two selected therefrom.

Wherein, the dyeing solution comprises fluorescein isothiocyanate and/or phycoerythrin, etc.

Wherein, the washing solution comprises phosphate buffer added with 1% bovine serum and/or phosphate buffer, etc.

In a preferred embodiment of the present application, steps (2′) to (3′) are successively repeated 1-20 time(s), for example 1 time, 2 times, 3 times, 4 times, 5 times, 6 times, 7 times, 8 times, 9 times, 10 times, 12 times, 15 times, 18 times, or 20 times, etc., before step (4′) is carried out. However, the repeated times are not limited to those specified above, and other un-specified values within the above ranges are also applicable.

In a preferred embodiment of the present application, steps (5′) to (6′) are successively repeated 1-20 time(s), for example 1 time, 2 times, 3 times, 4 times, 5 times, 6 times, 7 times, 8 times, 9 times, 10 times, 12 times, 15 times, 18 times or 20 times, etc., before step (7′) is carried out. However, the repeated times are not limited to those specified above, and other un-specified values within the above ranges are also applicable.

In a fifth aspect, the present application provides use of the microfluidic chip for separating circulating tumor cells as described in the first aspect or the method as described in the fourth aspect for counting circulating tumor cells.

In a sixth aspect, the present application provides use of the microfluidic chip for separating circulating tumor cells as described in the first aspect, the method as described in the second aspect, or the method as described in the fourth aspect for detecting circulating tumor cells.

The “first”, “second”, or the like as described in the present application are merely naming manners for clarity and convenience of description, and are not intended to limit the order of the structures so named and the order of use thereof.

Compared with the existing technical solutions, the present application has at least the following beneficial effects:

(1) The microfluidic chip for separating circulating tumor cells provided by the present application has the advantages of high throughput and high efficiency. With respect to the microfluidic chip, the filtration efficiency for blood cells can reach 99.99% or more, and the separation rate for circulating tumor cells can reach 86% or more.

(2) The method for separating circulating tumor cells provided by the present application, which uses the microfluidic chip for separating circulating tumor cells, greatly improves the efficiency of physical filtration and the purity of separated circulating tumor cells by adopting a manner of recirculating filtration.

(3) No expensive antibodies are required during the implementation of the method for separating circulating tumor cells as provided by the present application which uses the microfluidic chip for separating circulating tumor cells, resulting in a low cost.

### DESCRIPTION OF THE DRAWINGS

FIG. 1 shows the cross-sectional view of the structure of a microfluidic chip for separating circulating tumor cells provided by the present application;

FIG. 2 shows the schematic view of the structure of a microfluidic chip for separating circulating tumor cells provided by the present application.

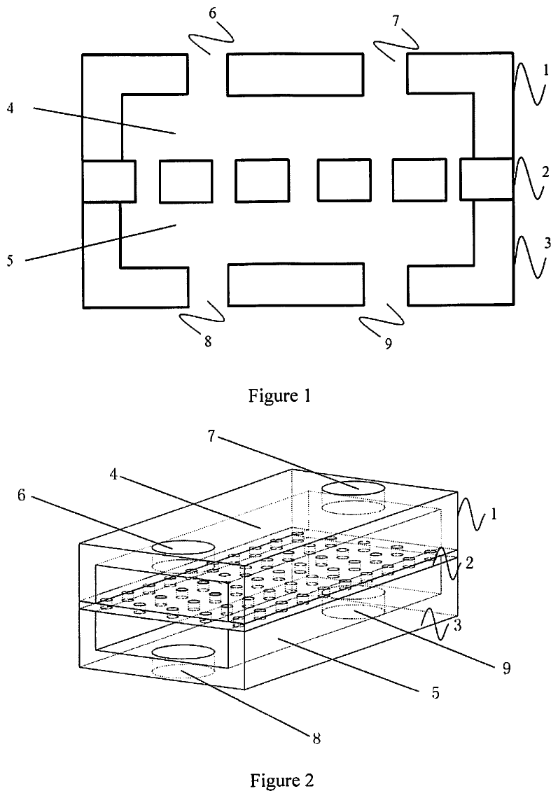

In FIGS. 1 and 2: 1—first housing layer, 2—filter membrane, 3—second housing layer, 4—first channel, 5—second channel, 6—inlet of the first housing layer, 7—outlet of the first housing layer, 8—inlet of the second housing layer, 9—outlet of the second housing layer.

### DETAILED DESCRIPTION

The technical solutions of the present application will be further described below with reference to the accompanying drawings and specific embodiments.

To better illustrate the present application and to facilitate understanding of the technical solutions of the present application, typical but non-limiting examples of the present application are set forth as follows.

### Example 1 Preparation of a Microfluidic Chip (I)

Provided was a microfluidic chip comprising a first housing layer, a second housing layer, and a filter membrane disposed between the first housing layer and the second housing layer, wherein a first channel was formed between the filter membrane and the first housing layer, and a second channel was formed between the filter membrane and the second housing layer;

the first housing layer was provided with m inlet(s) and n outlet(s), wherein m=3 and n=3;

the second housing layer was provided with x inlet(s) and y outlet(s), wherein x=2 and y=2;

the material of the first housing layer was dimethylsiloxane;

the material of the second housing layer was polymethylmethacrylate;

the material of the filter membrane was polymethylmethacrylate; and the filter membrane had a pore diameter of 10 μm.

### Example 2 Preparation of a Microfluidic Chip (II)

Provided was a microfluidic chip comprising a first housing layer, a second housing layer, and a filter membrane disposed between the first housing layer and the second housing layer, wherein a first channel was formed between the filter membrane and the first housing layer, and a second channel was formed between the filter membrane and the second housing layer;

the first housing layer was provided with m inlet(s) and n outlet(s), wherein m=1 and n=1;

the second housing layer was provided with x inlet(s) and y outlet(s), wherein x=1 and y=1;

the material of the first housing layer was polymethylmethacrylate;

the material of the second housing layer was polymethylmethacrylate;

the material of the filter membrane was polymethylmethacrylate; and the filter membrane had a pore diameter of 8 μm.

### Example 3 Preparation of a Microfluidic Chip (III)

Provided was a microfluidic chip comprising a first housing layer, a second housing layer, and a filter membrane disposed between the first housing layer and the second housing layer, wherein a first channel was formed between the filter membrane and the first housing layer, and a second channel was formed between the filter membrane and the second housing layer;

the first housing layer was provided with m inlet(s) and n outlet(s), wherein m=5 and n=4;

the second housing layer was provided with x inlet(s) and y outlet(s), wherein x=4 and y=5;

the material of the first housing layer was a combination of dimethylsiloxane and polymethylmethacrylate;

the material of the second housing layer was a combination of polymethylmethacrylate and polycarbonate;

the material of the filter membrane was a combination of polymethylmethacrylate and polycarbonate; and the filter membrane had a pore diameter of 9 μm.

### Example 4 Preparation of a Microfluidic Chip (IV)

Provided was a microfluidic chip comprising a first housing layer, a second housing layer, and a filter membrane disposed between the first housing layer and the second housing layer, wherein a first channel was formed between the filter membrane and the first housing layer, and a second channel was formed between the filter membrane and the second housing layer;

the first housing layer was provided with m inlet(s) and n outlet(s), wherein m=3 and n=5;

the second housing layer was provided with x inlet(s) and y outlet(s), wherein x=5 and y=3;

the material of the first housing layer was a combination of polycarbonate and dimethylsiloxane;

the material of the second housing layer was a combination of dimethylsiloxane, polymethylmethacrylate, and polycarbonate;

the material of the filter membrane was polycarbonate; and the filter membrane had a pore diameter of 15 μm.

### Example 5 Preparation of a Microfluidic Chip (V)

Provided was a microfluidic chip comprising a first housing layer, a second housing layer, and a filter membrane disposed between the first housing layer and the second housing layer, wherein a first channel was formed between the filter membrane and the first housing layer, and a second channel was formed between the filter membrane and the second housing layer;

the first housing layer was provided with m inlet(s) and n outlet(s), wherein m=2 and n=4;

the second housing layer was provided with x inlet(s) and y outlet(s), wherein x=2 and y=3;

the material of the first housing layer was polycarbonate;

the material of the second housing layer was polymethylmethacrylate;

the material of the filter membrane was dimethylsiloxane; and the filter membrane had a pore diameter of 7 μm.

### Example 6

Provided was a method for separating circulating tumor cells by using the microfluidic chip for separating circulating tumor cells, comprising the following steps:

(1) The inlet(s) of the first housing layer and the outlet(s) of the second housing layer were opened, and the outlet(s) of the first housing layer and the inlet(s) of the second housing layer were closed. Human peripherally circulating blood was diluted 5 times with phosphate buffer. 1 ml of the diluted sample was input via the inlet(s) of the first housing layer at a pressure of 40 mbar, filtered, and discharged via the outlet(s) of the second housing layer.

(2) The inlet(s) of the second housing layer and the outlet(s) of the second housing layer were opened, and the outlet(s) of the first housing layer and the inlet(s) of the first housing layer were closed. 800 μL of phosphate buffer was input via the inlet(s) of the second housing layer, and discharged via the outlet(s) of the second housing layer.

(3) The inlet(s) of the second housing layer and the outlet(s) of the first housing layer were opened, and the outlet(s) of the second housing layer and the inlet(s) of the first housing layer were closed. 800 μL of phosphate buffer was input via the inlet(s) of the second housing layer, and discharged via the outlet(s) of the first housing layer.

(4) The inlet(s) of the first housing layer and the outlet(s) of the first housing layer were opened, and the outlet(s) of the second housing layer and the inlet(s) of the second housing layer were closed. 800 μL of phosphate buffer was input via the inlet(s) of the first housing layer, and discharged via the outlet(s) of the first housing layer.

(5) The inlet(s) of the first housing layer and the outlet(s) of the second housing layer were opened, and the outlet(s) of the first housing layer and the inlet(s) of the second housing layer were closed. 800 μL of phosphate buffer was input via the inlet(s) of the first housing layer, and discharged via the outlet(s) of the second housing layer.

After step (5), steps (2) to (5) as described above were successively repeated 9 times. Wherein, the filtration flow rate in step (1) was 51 mL/h, the filtration flow rate in step (2) was 48 mL/h, the filtration flow rate in step (3) was 48 mL/h, the filtration flow rate in step (4) was 48 mL/h, and the filtration flow rate in step (5) was 48 mL/h.

Before the test, 1 mL of blood sample was taken and detected by flow cytometry. The number of total particles in the sample was 1.11×10⁹. While after ten times of filtration, the number of particles in the collected sample was 1.09×10⁹. The filtration efficiency of ten times of filtration for blood cells in the sample was 99.99%.

### Example 7

Provided was a method for separating circulating tumor cells using the microfluidic chip for separating circulating tumor cells, which was the same as Example 1 except that after step (5), steps (2) to (5) as described above were not repeated.

After one time of filtration, the number of particles in the collected sample was 9.63×10⁸. The filtration efficiency of one time of filtration for blood cells in the sample was 86.79%.

### Example 8

Provided was a method for counting circulating tumor cells using the microfluidic chip for separating circulating tumor cells, which comprised the following steps:

(1′) The inlet(s) of the first housing layer and the outlet(s) of the second housing layer were opened, and the outlet(s) of the first housing layer and the inlet(s) of the second housing layer were closed. SK-BR-3 cells having a concentration of about 1×10⁶ cells/mL were was diluted 10,000 times with phosphate buffer. 600 μL of the sample was input via the inlet(s) of the first housing layer, filtered and discharged via the outlet(s) of the second housing layer.

(2′) The inlet(s) of the second housing layer and the outlet(s) of the second housing layer were opened, and the outlet(s) of the first housing layer and the inlet(s) of the first housing layer were closed. 100 μL of phosphate buffer was input via the inlet(s) of the second housing layer, and discharged via the outlet(s) of the second housing layer.

(3′) The inlet(s) of the first housing layer and the outlet(s) of the second housing layer were opened, and the outlet(s) of the first housing layer and the inlet(s) of the second housing layer were closed. 100 μL of phosphate buffer was input via the inlet(s) of the first housing layer, and discharged via the outlet(s) of the second housing layer.

(4′) The inlets of the first housing layer were opened, and the outlets of the first housing layer, the inlets of the second housing layer and the outlets of the second housing layer were closed. A dye solution was input via the inlets of the first housing layer until the chip was filled with the dye solution. The chip was then allowed to stand.

(5′) The inlet(s) of the first housing layer and the outlet(s) of the second housing layer were opened, and the outlet(s) of the first housing layer and the inlet(s) of the second housing layer were closed. 100 μL of washing solution was input via the inlet(s) of the first housing layer, and discharged via the outlet(s) of the second housing layer.

(6′) The inlet(s) of the second housing layer and the outlet(s) of the first housing layer were opened, and the outlet(s) of the second housing layer and the inlet(s) of the first housing layer were closed. 100 μL of washing solution was input via the inlet(s) of the second housing layer, and discharged via the outlet(s) of the first housing layer.

(7′) Fluorescence signals from the stained cells inside the chip were observed under a fluorescence microscope and photographed for counting circulating tumor cells.

The number of separated SK-BR-3 cells was measured to be 56.

1 mL of un-filtered sample was subjected to counting of SK-BR-3 cells with a cytometer, and as a result, the number of SK-BR-3 cells was measured to be 7.561×10⁵. After one filtration and washing operation, the acquisition efficiency of the chip for tumor cells was 74.2%.

### Example 9

Provided was a method for counting circulating tumor cells using the microfluidic chip for separating circulating tumor cells, which was substantially the same as Example 3 except that steps (2′) to (3′) were successively repeated 20 times before step (4′) was carried out, and steps (5′) to (6′) were successively repeated 20 times before step (7′) was carried out.

The number of separated SK-BR-3 cells was measured to be 45.

After 20 times of filtration and washing operations, the acquisition efficiency of the chip for tumor cells was 59.6%.

### Example 10

A solution of SK-BR-3 cells having an average concentration of 1×10⁵ to 1×10⁶ cells/mL was diluted 10 times with phosphate buffer. 1 mL of the diluted cell solution was added into 1 mL of blood sample and operations were carried out in accordance with the method of Example 2.

The separated sample and un-filtrated tumor cell sample were counted respectively with a cell counter. The acquisition efficiency of the chip for SK-BR-3 cells was calculated to be 85.72%. After the treatment, the ratio of the number of tumor cells to that of blood cells was increased by 60 times.

### Example 11

A solution of SK-BR-3 cells having an average concentration of 1×10⁵ to 1×10⁶ cells/mL was diluted 10 times with phosphate buffer. 1 mL of the diluted cell solution was then added into 1 mL of blood sample and operations were carried out in accordance with the method of Example 4.

The un-filtrated tumor cell sample was counted with a cell counter. According to the counting results, the acquisition efficiency of the chip for SK-BR-3 cells was calculated to be 59.6%. After the treatment, the ratio of the number of tumor cells to that of blood cells was increased by 455 times.

### Comparative Example 1

Provided was a method for separating circulating tumor cells using the microfluidic chip for separating circulating tumor cells, which was substantially the same as Example 1 except that only step (1) was carried out.

The separated sample and un-filtrated sample were counted respectively with a cell counter. The acquisition efficiency of the chip for blood cells was calculated to be 43%.

### Comparative Example 2

Provided was a method for counting circulating tumor cells using the microfluidic chip for separating circulating tumor cells, which was substantially the same as Example 3 except that only step (1′) was carried out.

The separated sample and un-filtrated tumor cell sample were counted respectively with a cell counter. The acquisition efficiency of the chip for SK-BR-3 cells was calculated to be 65%.

### Comparative Example 3

A blood sample containing breast cancer cells was tested using the CellSearch system available from Johnson & Johnson according to the method as described in Lin, H. K., Zheng, S., Williams, A. J., Balic. M., Groshen, S., Scher, H. I., Cote, R. J. (2010). Portable filter-based microdevice for detection and characterization of circulating tumor cells. Clinical Cancer Research, 16(20), 5011-5018. The average acquisition efficiency for tumor cells was measured to be 13%.

It can be seen from Examples 6-11 that by using the microfluidic chip for separating circulating tumor cells and the method as provided by the present application, the acquisition efficiency for blood cells in a blood sample reached 99.99%, and the separation efficiency for cancer cells was also greater than 50%. For a mixture sample of cancer cells and blood, the ratio of the separated cancer cells to the residual blood cells was 455 times the initial ratio. In both Comparative Example 1 and Comparative Example 2, tumor cells were not separated according to the method as provided herein, and the separation efficiencies for both cancer cells and blood cells were decreased.

The applicant states that detailed structures of the present application are demonstrated in the present application through the above embodiments. However, the present application is not limited to the above detailed structures, and it does not mean that the present application must rely on the above detailed structures to implement. It should be apparent to those skilled in the art that, for any improvement of the present application, the equivalent replacement of the parts selected in the present application, the addition of auxiliary parts, and the selection of specific modes, etc., will all fall within the scope of protection and disclosure of the present application.

The preferred embodiments of the present application have been described in detail above. However, the present application is not limited to the specific details in the foregoing embodiments, and various simple modifications may be made to the technical solutions of the present application within the technical concept of the present application. These simple variants all fall within the scope of protection of the present application.

In addition, it should be noted that the specific technical features described in the above specific embodiments may be combined in any suitable manner without contradiction. In order to avoid unnecessary repetition, the present application will not be further described in various possible combinations. In addition, any combination of various embodiments of the present application may be made as long as it does not contradict the idea of the present application, and it should also be regarded as the disclosure of the present application.

1. A microfluidic chip for separating circulating tumor cells, comprising a first housing layer, a second housing layer, and a filter membrane disposed between the first housing layer and the second housing layer, wherein a first channel is formed between the filter membrane and the first housing layer, and a second channel is formed between the filter membrane and the second housing layer;
the first housing layer is provided with m inlet(s) and n outlet(s), wherein m≥1 and n≥1;
the second housing layer is provided with x inlet(s) and y outlet(s), wherein x≥1 and y≥1.

2. The microfluidic chip according to claim 1, wherein the material of the first housing layer comprises any one selected from the group consisting of dimethylsiloxane, polymethylmethacrylate, or polycarbonate, or a combination of at least two selected therefrom.

3. The microfluidic chip according to claim 1, wherein the material of the second housing layer comprises any one selected from the group consisting of dimethylsiloxane, polymethylmethacrylate, or polycarbonate, or a combination of at least two selected therefrom.

4. The microfluidic chip according to claim 1, wherein the material of the filter membrane comprises any one selected from the group consisting of dimethylsiloxane, polymethylmethacrylate, or polycarbonate, or a combination of at least two selected therefrom.

5. The microfluidic chip according to claim 1, wherein the filter membrane has a pore diameter of 7˜15 μm.

6. The microfluidic chip according to claim 1, wherein the filter membrane has a pore diameter of 8˜10 μm.

7. A method for separating circulating tumor cells by using the microfluidic chip for separating circulating tumor cells according to claim 1, comprising the following steps:
(1) opening the inlet(s) of the first housing layer and the outlet(s) of the second housing layer, closing the outlet(s) of the first housing layer and the inlet(s) of the second housing layer, and inputting a blood sample via the inlet(s) of the first housing layer, filtering the same, and discharging the filtrate via the outlet(s) of the second housing layer;
(2) opening the inlet(s) of the second housing layer and the outlet(s) of the second housing layer, closing the outlet(s) of the first housing layer and the inlet(s) of the first housing layer, and inputting a buffer via the inlet(s) of the second housing layer, and discharging outflow via the outlet(s) of the second housing layer;
(3) opening the inlet(s) of the second housing layer and the outlet(s) of the first housing layer, closing the outlet(s) of the second housing layer and the inlet(s) of the first housing layer, and inputting a buffer via the inlet(s) of the second housing layer, and discharging outflow via the outlet(s) of the first housing layer;
(4) opening the inlet(s) of the first housing layer and the outlet(s) of the first housing layer, closing the outlet(s) of the second housing layer and the inlet(s) of the second housing layer, and inputting a buffer via the inlet(s) of the first housing layer, and discharging outflow via the outlet(s) of the first housing layer;
(5) opening the inlet(s) of the first housing layer and the outlet(s) of the second housing layer, closing the outlet(s) of the first housing layer and the inlet(s) of the second housing layer, and inputting a buffer via the inlet(s) of the first housing layer, and discharging outflow via the outlet(s) of the second housing layer.

8. The method according to claim 7, wherein steps (2) to (5) are repeated successively 1-20 time(s).

9. (canceled)

10. A method for counting circulating tumor cells by using the microfluidic chip for separating circulating tumor cells according to claim 1, comprising the following steps:
(1′) opening the inlet(s) of the first housing layer and the outlet(s) of the second housing layer, closing the outlet(s) of the first housing layer and the inlet(s) of the second housing layer, and inputting a blood sample via the inlet(s) of the first housing layer, filtering the same, and discharging the filtrate via the outlet(s) of the second housing layer;
(2′) opening the inlet(s) of the second housing layer and the outlet(s) of the second housing layer, closing the outlet(s) of the first housing layer and the inlet(s) of the first housing layer, and inputting a buffer via the inlet(s) of the second housing layer, and discharging outflow via the outlet(s) of the second housing layer;
(3′) opening the inlet(s) of the first housing layer and the outlet(s) of the second housing layer, closing the outlet(s) of the first housing layer and the inlet(s) of the second housing layer, and inputting a buffer via the inlet(s) of the first housing layer, and discharging outflow via the outlet(s) of the second housing layer;
(4′) opening the inlet(s) of the first housing layer, closing the outlet(s) of the first housing layer, the inlet(s) of the second housing layer and the outlet(s) of the second housing layer, inputting a dyeing solution via the inlet(s) of the first housing layer until the chip is filled with the dyeing solution, and allowing the chip to stand;
(5′) opening the inlet(s) of the first housing layer and the outlet(s) of the second housing layer, closing the outlet(s) of the first housing layer and the inlet(s) of the second housing layer, and inputting a washing solution via the inlet(s) of the first housing layer, and discharging outflow via the outlet(s) of the second housing layer;
(6′) opening the inlet(s) of the second housing layer and the outlet(s) of the first housing layer, closing the outlet(s) of the second housing layer and the inlet(s) of the first housing layer, and inputting a washing solution via the inlet(s) of the second housing layer, and discharging outflow via the outlet(s) of the first housing layer;
(7′) observing fluorescence signals from the stained cells inside the chip under a fluorescence microscope, and photographing the same for counting circulating tumor cells.

11. The method according to claim 10, wherein steps (2′) to (3′) are successively repeated 1-20 time(s) before step (4′) is carried out.

12. The method according to claim 10, wherein steps (5′) to (6′) are successively repeated 1-20 time(s) before step (7′) is carried out.

13. (canceled)

14. (canceled)
